SEQUENCE LISTING (49 sequences)

SEQ ID NO: 1 - protein, 8 aa, Mouse
  18G7.1 Heavy Chain CDR1 Amino Acid
GYTFSGYW

SEQ ID NO: 2 - protein, 8 aa, Mouse
  18G7.1 Heavy Chain CDR2 Amino Acid
ILPGSDST

SEQ ID NO: 3 - protein, 11 aa, Mouse
  18G7.1 Heavy Chain CDR3 Amino Acid
ARSGYYGSSQY

SEQ ID NO: 4 - protein, 10 aa, Mouse
  18G7.1 Light Chain CDR1 Amino Acid
ESVDSYGNSF

SEQ ID NO: 5 - protein, 3 aa, Mouse
  18G7.1 Light Chain CDR2 Amino Acid
LTS

SEQ ID NO: 6 - protein, 10 aa, Mouse
  18G7.1 Light Chain CDR3 Amino Acid
MQQNNEDPRT

SEQ ID NO: 7 - DNA, 354 bp, Artifical Sequence
  18G7H6A1 Heavy Chain DNA
gaggtgcagctggtgcagagcggagccgaggtgaagaagcccggcgagagcctgaggatcagctgcaagggcagcggctacagcttcaccgcgtactggatcgagtgggtgaggcaggctcccggcaagggcctggagtggatcggcgagatcctgcccggcagcgacagcaccaactacaacgagaagttcaagggccacgtgaccatcagcgccgacaagagcatcagcaccgcctacctgcagtggagcagcctgaaggccagcgacaccgccgtgtactactgcgcccgcagcggctactacggcagcagccagtactggggccagggcaccctggtgaccgtgagcagc

SEQ ID NO: 8 - DNA, 333 bp, Artificial Sequence
  18G7H6A1 Light Chain DNA
gacatcgtgctgacccagagccccgccagcctggccgtgagccccggccagagggccaccatcacctgccgcgccagcgagagcgtggacagctacggcaacagcttcatgcactggtatcagcagaagcccggccagccccccaagctgctgatctacctgaccagcaacctggagtccggcgtgcccgacaggttcagcggcagcggcagcggcaccgacttcaccctgaccatcaaccccgtggaggccaacgacgccgccacctactactgccagcagaacgccgaggaccccaggaccttcggcggcggcaccaagctggagatcaag

SEQ ID NO: 9 - protein, 466 aa, Artificial Sequence
  18G7H6A1 Heavy Chain Amino Acid
MEWSWVFLFFLSVTTGVHSEVQLVQSGAEVKKPGESLRISCKGSGYSFTAYWIEWVRQAPGKGLEWIGEILPGSDSTNYNEKFKGHVTISADKSISTAYLQWSSLKASDTAVYYCARSGYYGSSQYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 238 aa, Artificial Sequence
  18G7H6A1 Light Chain Amino Acid
MSVPTQVLGLLLLWLTDARCDIVLTQSPASLAVSPGQRATITCRASESVDSYGNSFMHWYQQKPGQPPKLLIYLTSNLESGVPDRFSGSGSGTDFTLTINPVEANDAATYYCQQNAEDPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 1423 bp, Artificial Sequence
  18G7H6A3 Heavy Chain DNA
aagcttgccgccaccatggaatggtcctgggtgttcctgttcttcctgtccgtgaccaccggcgtgcactccgaagtgcagctggtgcagtctggcgccgaagtgaagaagcctggcgagtccctgcggatctcctgcaagggctccggctactccttcaccgcctactggattgagtgggtgcgacaggcccctggcaagggcctggaatggatcggagagatcctgcccggctccgactccaccaactacaacgagaagttcaagggccacgtgaccatctccgccgacaagtccatctctaccgcctacctgcagtggtcctccctgaaggcctctgacaccgccgtgtactactgcgccagatccggcctgtacggctcctctcagtattggggccagggcaccctcgtgaccgtgtcctctgcttctaccaagggcccaagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgcccagcccccgagctgctgggcggacccagcgtgttcctgttcccccccaagcccaaggacaccctgatgatcagcagaacccccgaggtgacctgtgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggagtacaagtgtaaggtgtccaacaaggccctgccagccccaatcgaaaagaccatcagcaaggccaagggccagccaagagagccccaggtgtacaccctgccacccagcagggacgagctgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaagctgaccgtggacaagagcagatggcagcagggcaacgtgttcagctgctccgtgatgcacgaggccctgcacaaccactacacccagaagagcctgagcctgtccccaggctgatgaattc

SEQ ID NO: 12 - DNA, 739 bp, Artificial Sequence
  18G7H6A3 Light Chain DNA
aagcttgccgccaccatgtccgtgcctacccaggtgctgggactgctgctgctgtggctgaccgacgccagatgcgacatcgtgctgacccagagccctgcctctctggctgtgtctcctggccagagggccaccatcacctgtagagcctccgagtccgtggactcctacggcaactccttcatgcactggtatcagcagaagcccggccagccccccaagctgctgatctacctgacctccaacctggaatccggcgtgcccgacagattctccggctctggctctggcaccgacttcaccctgaccatcaaccccgtggaagccaacgacgccgccacctactactgccagcagaacgccgaggaccccagaacctttggcggaggcaccaagctggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgctgatgaattc

SEQ ID NO: 13 - protein, 466 aa, Artificial Sequence
  18G7H6A3 Heavy Chain Amino Acid
MEWSWVFLFFLSVTTGVHSEVQLVQSGAEVKKPGESLRISCKGSGYSFTAYWIEWVRQAPGKGLEWIGEILPGSDSTNYNEKFKGHVTISADKSISTAYLQWSSLKASDTAVYYCARSGLYGSSQYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 238 aa, Artificial Sequence; identical to SEQ ID NO: 10
  18G7H6A3 Light Chain Amino Acid

SEQ ID NO: 15 - DNA, 354 bp, Artificial Sequence
  18G7Ch Heavy Chain DNA
caggttcagctgcagcagtctggagctgagctggtgaagcctggggcctcagtgaagatatcctgcaaggctactggctacacattcagtggctactggatagagtgggtaaagcagaggcctggacatggccttgagtggattggagagattttgcctggaagtgatagtactaactacaatgagaagttcaagggcaaggccacattcactgcagatacatcctccaacacagtctacatgcaattcagcagcctgacatctgaggactctgccgtctattactgtgcaagatcgggttactacggtagtagtcagtactggggccaaggcaccactctcacagtctcctca

SEQ ID NO: 16 - DNA, 334 bp, Artificial Sequence
  18G7Ch Light Chain DNA
aacattgtgctgacccaatctcctgcttctttggctgtgtctctagggcagagggccaccatatcctgcagagccagtgaaagtgttgatagttatggcaatagttttatgcactggtaccagcagaaaccaggacagccacccaaactcctcatctatcttacatccaacctagaatctggggtccctgccaggttcagtggcagtgggtctaggacagacttcaccctcaccattgatcctgtggaggctgatgatgctgcaacctattactgtcagcaaaataatgaggatcctcggacgttcggtggaggcaccaagctggaaatcaaac

SEQ ID NO: 17 - protein, 466 aa, Artificial Sequence
  18G7Ch Heavy Chain Amino Acid
MEWSWVFLFFLSVTTGVHSQVQLQQSGAELVKPGASVKISCKATGYTFSGYWIEWVKQRPGHGLEWIGEILPGSDSTNYNEKFKGKATFTADTSSNTVYMQFSSLTSEDSAVYYCARSGYYGSSQYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 238 aa, Artificial Sequence
  18G7ch Light Chain Amino Acid
MSVPTQVLGLLLLWLTDARCNIVLTQSPASLAVSLGQRATISCRASESVDSYGNSFMHWYQQKPGQPPKLLIYLTSNLESGVPARFSGSGSRTDFTLTIDPVEADDAATYYCQQNNEDPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 137 aa, Artificial Sequence
  18G7H6A3 Heavy Chain Variable Domain Amino Acid
MEWSWVFLFFLSVTTGVHSEVQLVQSGAEVKKPGESLRISCKGSGYSFTAYWIEWVRQAPGKGLEWIGEILPGSDSTNYNEKFKGHVTISADKSISTAYLQWSSLKASDTAVYYCARSGYYGSSQYWGQGTLVTVSS

SEQ ID NO: 20 - DNA, 411 bp, Artificial Sequence
  18G7H6A3 Heavy Chain Variable Domain DNA
atggaatggtcctgggtgttcctgttcttcctgtccgtgaccaccggcgtgcactccgaagtgcagctggtgcagtctggcgccgaagtgaagaagcctggcgagtccctgcggatctcctgcaagggctccggctactccttcaccgcctactggattgagtgggtgcgacaggcccctggcaagggcctggaatggatcggagagatcctgcccggctccgactccaccaactacaacgagaagttcaagggccacgtgaccatctccgccgacaagtccatctctaccgcctacctgcagtggtcctccctgaaggcctctgacaccgccgtgtactactgcgccagatccggcctgtacggctcctctcagtattggggccagggcaccctcgtgaccgtgtcctct

SEQ ID NO: 21 - protein, 131 aa, Artificial Sequence
  18G7H6A3 Light Chain Variable Domain
MSVPTQVLGLLLLWLTDARCDIVLTQSPASLAVSPGQRATITCRASESVDSYGNSFMHWYQQKPGQPPKLLIYLTSNLESGVPDRFSGSGSGTDFTLTINPVEANDAATYYCQQNAEDPRTFGGGTKLEIK

SEQ ID NO: 22 - DNA, 393 bp, Artificial Sequence
  18G7H6A3 Light Chain Variable Domain DNA
atgtccgtgcctacccaggtgctgggactgctgctgctgtggctgaccgacgccagatgcgacatcgtgctgacccagagccctgcctctctggctgtgtctcctggccagagggccaccatcacctgtagagcctccgagtccgtggactcctacggcaactccttcatgcactggtatcagcagaagcccggccagccccccaagctgctgatctacctgacctccaacctggaatccggcgtgcccgacagattctccggctctggctctggcaccgacttcaccctgaccatcaaccccgtggaagccaacgacgccgccacctactactgccagcagaacgccgaggaccccagaacctttggcggaggcaccaagctggaaatcaag

SEQ ID NO: 23 - protein, 8 aa, Artificial Sequence
  18G7H6A3 Heavy Chain CDR1 Amino Acid
GYSFTAYW

SEQ ID NO: 24 - DNA, 24 bp, Artificial Sequence
  18G7H6A3 Heavy Chain CDR1 DNA
ggctactccttcaccgcctactgg

SEQ ID NO: 25 - protein, 8 aa, Artificial Sequence
  18G7H6A3 Heavy Chain CDR2 Amino Acid
ILPGSDST

SEQ ID NO: 26 - DNA, 24 bp, Artificial Sequence
  18G7H6A3 Heavy Chain CDR2 DNA
atcctgcccggctccgactccacc

SEQ ID NO: 27 - protein, 11 aa, Artificial Sequence
  18G7H6A3 Heavy Chain CDR3 Amino Acid
ARSGYYGSSQY

SEQ ID NO: 28 - DNA, 33 bp, Artificial Sequence
  18G7H6A3 Heavy Chain CDR3 DNA
gccagatccggcctgtacggctcctctcagtat

SEQ ID NO: 29 - protein, 10 aa, Artificial Sequence
  18G7H6A3 Light Chain CDR1 Amino Acid
ESVDSYGNSF

SEQ ID NO: 30 - DNA, 30 bp, Artificial Sequence
  18G7H6A3 Light Chain CDR1 DNA
gagtccgtggactcctacggcaactccttc

SEQ ID NO: 31 - protein, 3 aa, Artificial Sequence
  18G7H6A3 Light Chain CDR2 Amino Acid
LTS

SEQ ID NO: 32 - DNA, 9 bp, Artificial Sequence
  18G7H6A3 Light Chain CDR2 DNA
ctgacctcc

SEQ ID NO: 33 - protein, 9 aa, Artificial Sequence
  18G7H6A3 Light Chain CDR3 Amino Acid
QQNAEDPRT

SEQ ID NO: 34 - DNA, 27 bp, Artificial Sequence
  18G7H6A3 Light Chain CDR3 DNA
cagcagaacgccgaggaccccagaacc

SEQ ID NO: 35 - protein, 8 aa, Artificial Sequence
  18G7H6A1 Heavy Chain CDR1 Amino Acid
GYSFTAYW

SEQ ID NO: 36 - DNA, 24 bp, Artificial Sequence
  18G7H6A1 Heavy Chain CDR1 DNA
ggctactccttcaccgcctactgg

SEQ ID NO: 37 - protein, 8 aa, Artificial Sequence
  18G7H6A1 Heavy Chain CDR2 Amino Acid
ILPGSDST

SEQ ID NO: 38 - DNA, 24 bp, Artificial Sequence
  18G7H6A1 Heavy Chain CDR2 DNA
atcctgcccggcagcgacagcacc

SEQ ID NO: 39 - protein, 11 aa, Artificial Sequence
  18G7H6A1 Heavy Chain CDR3 Amino Acid
ARSGYYGSSQY

SEQ ID NO: 40 - DNA, 33 bp, Artificial Sequence
  18G7H6A1 Heavy Chain CDR3 DNA
gcccgcagcggctactacggcagcagccagtac

SEQ ID NO: 41 - protein, 10 aa, Artificial Sequence
  18G7H6A1 Light Chain CDR1 Amino Acid
ESVDSYGNSF

SEQ ID NO: 42 - DNA, 30 bp, Artificial Sequence
  18G7H6A1 Light Chain CDR1 DNA
gagagcgtggacagctacggcaacagcttc

SEQ ID NO: 43 - protein, 3 aa, Artificial Sequence
  18G7H6A1 Light Chain CDR2 Amino Acid
LTS

SEQ ID NO: 44 - DNA, 9 bp, Artificial Sequence
  18G7H6A1 Light Chain CDR2 DNA
ctgaccagc

SEQ ID NO: 45 - protein, 9 aa, Artificial Sequence
  18G7H6A1 Light Chain CDR3 Amino Acid
QQNAEDPRT

SEQ ID NO: 46 - DNA, 27 bp, Artificial Sequence
  18G7H6A1 Light Chain CDR3 DNA
cagcagaacgccgaggaccccaggacc

SEQ ID NO: 47 - protein, 561 aa, Homo Sapiens
MDTSRLGVLLSLPVLLQLATGGSSPRSGVLLRGCPTHCHCEPDGRMLLRVDCSDLGLSELPSNLSVFTSYLDLSMNNISQLLPNPLPSLRFLEELRLAGNALTYIPKGAFTGLYSLKVLMLQNNQLRHVPTEALQNLRSLQSLRLDANHISYVPPSCFSGLHSLRHLWLDDNALTEIPVQAFRSLSALQAMTLALNKIHHIPDYAFGNLSSLVVLHLHNNRIHSLGKKCFDGLHSLETLDLNYNNLDEFPTAIRTLSNLKELGFHSNNIRSIPEKAFVGNPSLITIHFYDNPIQFVGRSAFQHLPELRTLTLNGASQITEFPDLTGTANLESLTLTGAQISSLPQTVCNQLPNLQVLDLSYNLLEDLPSFSVCQKLQKIDLRHNEIYEIKVDTFQQLLSLRSLNLAWNKIAIIHPNAFSTLPSLIKLDLSSNLLSSFPITGLHGLTHLKLTGNHALQSLISSENFPELKVIEMPYAYQCCAFGVCENAYKISNQWNKGDNSSMDDLHKKDAGMFQAQDERDLEDFLLDFEEDLKALHSVQCSPSPGPFKPCEHLLDGWLIR

SEQ ID NO: 48 - protein, 137 aa, Artificial Sequence; identical to SEQ ID NO: 19
  18G7H6A1 Heavy Chain Variable Amino acid

SEQ ID NO: 49 - protein, 131 aa, Artificial Sequence; identical to SEQ ID NO: 21
  18G7H6A1 Light Chain Variable Amino acid

(19) United States
(12) Patent Application Publication
(10) Pub. No.: US 2020/0114017 A1
(43) Pub. Date: Apr. 16, 2020

(54) ANTIBODY DRUG CONJUGATES THAT BIND LGR5

(71) Applicant: Bionomics Limited, Thebarton, South Australia (AU)

(72) Inventors: Christopher L. Reyes, San Diego, CA (US); Peter Chu, San Diego, CA (US)

(21) Appl. No.: 16/621,897

(22) PCT Filed: Jun. 16, 2017
(86) PCT No.: PCT/US2018/037613

§ 371 Date: Dec. 12, 2019

Related U.S. Application Data
(60) Provisional application No. 62/520,726, filed on Jun. 16, 2017.

(51) Int. Cl. A61K 47/68; A61P 35/00; C07K 16/28

(52) U.S. Cl. CPC A61K 47/6851; A61K 47/6803; A61K 45/06; A61P 35/00; C07K 16/28; A61K 47/6849

(57) ABSTRACT

Embodiments of the methods and compositions provided herein relate to antibody drug conjugates comprising an antibody or antigen binding fragment thereof that specifically binds to human LGR5 in which the antibody or antigen binding fragment thereof is conjugated to a therapeutic agent, such as a drug, via a linker. Some embodiments relate to methods of treatment using such antibody drug conjugates, and methods of preparing such antibody drug conjugates.

### RELATED APPLICATIONS

This application claims priority to U.S. Prov. App. No. 62/520,726 filed Jun. 16, 2017 which is incorporated herein in its entirety.

### FIELD OF THE INVENTION

Embodiments of the methods and compositions provided herein relate to antibody drug conjugates (ADCs) comprising an antibody or antigen binding fragment thereof that specifically binds to human LGR5 in which the antibody or antigen binding fragment thereof is conjugated to a therapeutic agent, such as a drug, via a linker. Some embodiments relate to methods of treatment using such ADCs, and methods of preparing such ADCs.

### REFERENCE TO SEQUENCE LISTING

The present application is being filed along with a Sequence Listing in electronic format. The Sequence Listing is provided as a file entitled BIONO15WOSEQLISTING.TXT, created Jun. 7, 2018 which is approximately 40 Kb in size. The information in the electronic format of the Sequence Listing is incorporated herein by reference in its entirety.

### BACKGROUND OF THE INVENTION

Leucine-rich repeat containing G-protein-coupled receptor 5 (LGR5), also known as GPR49/HG38/FEX, belongs to the leucine-rich repeat containing G-protein-coupled receptor (LGR)/G-Protein-coupled Receptor (GPR) protein family of receptor proteins that are structurally similar to glycoprotein hormone receptors. LGRs are divided into three subgroups: (1) glycoprotein hormone receptors including thyroid-stimulating hormone (TSH) receptor, follicle-stimulating hormone (FSH) receptor, and luteinizing hormone (LH) receptor; (2) relaxin receptors LGR7 and LGR8; and (3) LRG4, LGR5, and LGR6. LGR5 is expressed in several tissues including the intestine, skeletal muscle, placenta, brain, and spinal cord.

### SUMMARY OF THE INVENTION

Some embodiments of the methods and compositions provided herein include an antibody drug conjugate comprising an antibody or antigen-binding fragment thereof that specifically binds to human leucine-rich repeat containing G-protein-coupled receptor 5 (LGR5), wherein the antibody or antigen-binding fragment thereof is conjugated to a drug via a linker.

In some embodiments, the antibody or antigen-binding fragment thereof comprises: a heavy chain complementary determining region (CDR1) comprising SEQ ID NO:23, or conservative variations thereof, a heavy chain complementary determining region 2 (CDR2) comprising SEQ ID NO:25, or conservative variations thereof, a heavy chain complementary determining region 3 (CDR3) comprising SEQ ID NO:27, or conservative variations thereof, a light chain CDR1 comprising SEQ ID NO:29, or conservative variations thereof a light chain CDR2 comprising SEQ ID NO:31, or conservative variations thereof, and a light chain CDR3 comprising SEQ ID NO:33, or conservative variations thereof. In some embodiments, the antibody or antigen-binding fragment thereof comprises a heavy chain CDR1 comprising SEQ ID NO:23. In some embodiments, the anti-LGR5 antibody or antigen-binding fragment thereof comprises an IgG1.

In some embodiments, the linker is a non-cleavable linker. In some embodiments, the linker is a cleavable linker.

In some embodiments, the drug is selected from a microtubulin inhibitor and a DNA damaging agent. In some embodiments, the microtubule inhibitor is selected form the group consisting of cabazitaxel, colcemid, colchicine, cryptophycin, demecolcine, docetaxel, 2-Methoxyestradiol, docodazole, paclitaxel, taccalonolide, taxane, and vinblastine. In some embodiments, the drug is selected from the group consisting of monomethyl auristatin F, monomethyl auristatin E, monomethyl dolastatin 10, duocarmycin, maytansanoid 1, dualstatin 3, calicheamicin, and duocamycin.

Some embodiments of the methods and compositions provided herein a pharmaceutical composition comprising the antibody drug conjugate provided herein, and a pharmaceutically acceptable carrier.

Some embodiments of the methods and compositions provided herein a method of treating a subject having a cancer comprising administering an effective amount of the antibody drug conjugate provided herein to the subject in need thereof. In some embodiments, the cancer comprises a solid tumor. In some embodiments, the cancer comprises a cancer stem cell. In some embodiments, the cancer is selected from the group consisting of: lung cancer, breast cancer, colon cancer, and pancreatic cancer. In some embodiments, the cancer comprises a cell selected from the group consisting of: a triple negative breast cancer cell, a colon cancer cell having a mutation in a gene selected from the group consisting of K-Ras, H-Ras, APC, PI3K, PTEN, STK11, RB1, TP53, FGFR2, VANGL2, and ISCO, and a small cell lung cancer cell. In some embodiments, the subject is mammalian. In some embodiments, the subject is human.

Some embodiments also include administering an additional therapy in combination with the antibody drug conjugate, wherein the additional therapy is selected from the group consisting of: radiotherapy, and a chemotherapeutic agent. In some embodiments, administration of the antibody drug conjugate is concurrent with administration of the additional therapy In some embodiments, the chemotherapeutic agent is selected from the group consisting of: folinic acid, fluorouracil, irinotecan, gemcitabine, paclitaxel, nab-paclitaxel, ERBITUX (cetuximab), PI3K/mTOR dual inhibitor (NVP), and SN38. In some embodiments, the additional therapy comprises folinic acid, fluorouracil, and irinotecan.

Some embodiments of the methods and compositions provided herein a method of preparing the antibody drug conjugate provided herein include: linking the linker to the drug; and conjugating the linked drug to the antibody. Some embodiments also include purifying the conjugated antibody.

### BRIEF DESCRIPTION OF THE DRAWINGS

FIG. 1A depicts a graph of cell viability for cells treated with a primary anti-LGR5 antibody (C12) and a secondary ADC conjugated with NC-MMAF. Mean +/−SEM, n=2.

FIG. 1B depicts a graph of cell viability for cells treated with a primary anti-LGR5 antibody (C12) and a secondary ADC conjugated with CL-MD10. Mean +/−SEM, n=2.

FIG. 1C depicts a graph of cell viability for cells treated with a primary anti-LGR5 antibody (C12) and a secondary ADC conjugated with CL-MMAE. Mean +/−SEM, n=2.

FIG. 1D depicts a graph of cell viability for cells treated with a primary anti-LGR5 antibody (C12) and a secondary ADC conjugated with CL-DMSA. Mean +/−SEM, n=2.

FIG. 1E depicts a graph of cell viability for cells treated with a primary anti-LGR5 antibody (C12) and a secondary ADC conjugated with NC-DM1. Mean +/−SEM, n=2.

FIG. 1F depicts a graph of cell viability for cells treated with a primary anti-LGR5 antibody (C12) and a secondary ADC conjugated with CK-DUA3. Mean +/−SEM, n=2.

FIG. 1G depicts a graph of cell viability for cells treated with a primary anti-LGR5 antibody (C12) and a secondary ADC conjugated with CK-CAL. Mean +/−SEM, n=2.

FIG. 2 depicts a series of graphs of cell viability for cells treated with different primary anti-LGR5 antibodies, and a secondary ADC conjugated with NC-MMAF. The different primary anti-LGR5 antibodies included: C12 (top left panel); 18G7Ch (top right panel); 18G7H6A1 (bottom left panel); and 18G7H6A3 (bottom right panel). Mean +/−SEM.

FIG. 3 depicts a series of graphs of tumorsphere formation for cells treated with different primary anti-LGR5 antibodies, and a secondary ADC conjugated with CL-DMSA. The different primary anti-LGR5 antibodies included: C12 (top left panel); 18G7Ch (top right panel); 18G7H6A1 (bottom left panel); and 18G7H6A3 (bottom right panel). Mean +/−SEM.

FIG. 4 depicts a series of graphs of tumorsphere formation for cells treated with primary anti-LGR5 antibodies conjugated with duocarmycin (left panel), or with MMAE (right panel). Mean +/−SEM.

FIG. 5A depicts a graph of tumor volume over time in a xenograft model treated with an anti-LGR5 antibody (BNC101) conjugated with MMAE, or PBS (n=6).

FIG. 5B depicts a graph of tumor volume over time in a xenograft model treated with an anti-LGR5 antibody (BNC101) conjugated with MMAE, MOPC-MMAE, or PBS (n=6).

FIG. 5C depicts a graph of tumor volume over time in a xenograft model treated with an anti-LGR5 antibody (BNC101) conjugated with duocarmycin, MOPC-duocarmycin, or PBS (n=6).

FIG. 6A depicts an example of a structure of a linker and a DMSA.

FIG. 6B depicts an example of a structure of a DMSA linked to an antibody.

### DETAILED DESCRIPTION

Embodiments of the methods and compositions provided herein relate to antibody drug conjugates (ADCs) comprising an antibody, or antigen binding fragment thereof, that specifically binds to human LGR5 and is conjugated to a therapeutic agent. The therapeutic agent may be a drug that is bound to the antibody or antigen binding fragment thereof via a linker. Some embodiments relate to methods of treatment using such ADCs, and methods of preparing such ADCs.

The following references each relate to antibodies that specifically bind to human LGR5 useful with the methods and compositions provided herein: U.S. Pat. Nos. 9,221,906; 9,220,774; 9,221,907; 9,546,214; and 9,631,024, each of which is expressly incorporated herein by reference in its entirety. For example, U.S. Pat. Nos. 9,221,906; 9,220,774; and 9,221,907 each disclose antibodies that specifically bind to human LGR5 useful with the methods and compositions provided herein. U.S. Pat. No. 9,546,214 discloses humanized antibodies that specifically bind to human LGR5 useful with the methods and compositions provided herein.

LGR5 was identified through lineage tracing studies as a highly specific marker of normal stem cells and tumor-initiating cells in the gut. Previously about 150 genes were identified whose expression was quenched following abrogation of Wnt expression. A comprehensive characterization of these ‘Wnt target genes’ found LGR5 to be selectively expressed on a population of 10-14 proliferating wedge-shaped cells at the crypt base. These crypt-based columnar cells were previously proposed to be a candidate stem cell population. Using in vivo lineage tracing with a heritable lacZ−LGR5 reporter gene, it has been confirmed that LGR5 intestinal stem cells are a multi-potent, self-renewing population of adult intestinal stem cells that give rise to uninterrupted ribbons of lacZ+ progeny cells initiating from the crypt base and extending to the villus tips.

The specific expression of LGR5 on cancer stem cells (CSCs) provides an opportunity to target CSCs selectively and effectively. LGR5 is highly over expressed in colorectal cancer (CRC), pancreatic and most other solid tumors, compared to normal tissues, thereby providing a wide therapeutic window to target CSCs in CRC, pancreatic, breast, ovarian, lung, gastric and liver cancer.

A functional role of LGR5 in cancer has been validated through ribonucleic acid interference (RNAi) knockdown studies. Knockdown of LGR5 in a panel of CRC tumor cell lines significantly inhibited the growth of soft agar colonies in vitro, and also the growth of HCT116 colon tumor xenografts in viva. LGR5 RNAi knockdown was subsequently shown to also reduce the growth of CSC colonies from patient-derived CRC tumor cells in vitro (data not shown). Finally, sorted LGR5 (+) patient derived xenograft CRC tumor cells were found to be highly tumorigenic in vivo compared to control LGR5 (−) cells.

CSCs are believed to responsible for the high incidence of tumor recurrence in many cancer patients treated with surgery and standard of care chemotherapy. For example, CD44+ CSCs from breast cancer patients were found to be enriched following chemotherapy, and that high levels of CSCs correlated with poor clinical response to chemotherapy. Similarly, in metastatic CRC, LGR5 expression was upregulated in damaged liver following chemotherapy, suggesting that increased LGR5 CSCs in response to chemotherapy initiate and/or acerbate metastatic disease. Indeed, it has been found that LGR5 expression is significantly greater in metastatic sites compared to primary CRC tumors.

### Anti-LGR5 Antibodies

Embodiments of the methods and compositions provided herein include antibodies or antigen binding fragments thereof that specifically bind to human LGR5. As used herein, the term “antibody” includes, but is not limited to, synthetic antibodies, monoclonal antibodies, recombinantly produced antibodies, intrabodies, multispecific antibodies (including bi-specific antibodies), human antibodies, humanized antibodies, chimeric antibodies, synthetic antibodies, single-chain Fvs (scFv), Fab fragments, F(ab′) fragments, disulfide-linked Fvs (sdFv) (including bi-specific sdFvs), and anti-idiotypic (anti-Id) antibodies, and epitope-binding or antigen-binding fragments of any of the above, in which the antigen is LGR5. The antibodies of several embodiments provided herein may be monospecific, bispecific, trispecific or of greater multispecificity. Multispecific antibodies may be specific for different epitopes of a polypeptide or may be specific for both a polypeptide as well as for a heterologous epitope, such as a heterologous polypeptide or solid support material. See, e.g., PCT publications WO 93/17715: WO 92/08802; WO91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992); each of which is incorporated herein by reference in its entirety.

As used herein, LGR5 includes, but is not limited to, human LGR5 including the polypeptide of NCBI Accession No. NP_003658.1, or fragments thereof, which is encoded by the coding nucleotide sequence within NM_003667.2, or fragments thereof. The amino acid sequence and entire entry of NCBI Accession No. NP_003658.1 and nucleotide sequence and entire entry of NM_003667.2 are fully incorporated by reference in their entireties. Examples of LGR5 fragments contemplated herein include the LGR5 ectodomain, transmembrane domain, or intracellular domain and portions thereof.

Some embodiments are drawn to a nucleic acid molecule encoding the light chain or the heavy chain of an anti-LGR5 antibody, including any one of the anti-LGR5 antibodies designated as 18G7Ch, 18G7H6A3 and 18G7H6A1 provided herein. In some aspects, a nucleic acid molecule encodes the light chain or the heavy chain of a humanized or fully human monoclonal, such as antibody 18G7Ch, 18G7H6A3 and 18G7H6A1 provided herein.

Various embodiments are directed to a vector comprising a nucleic acid molecule or molecules encoding a light chain and/or a heavy chain of an anti-LGR5 antibody, including any one of the anti-LGR5 antibodies designated as 18G7Ch, 18G7H6A3 and 18G7H6A1 provided herein.

In various embodiments, the glycosylation of the antibodies can be modified. For example, an aglycosylated antibody can be made (i.e., the antibody lacks glycosylation). Glycosylation can be altered to, for example, increase the affinity of the antibody for a target antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861; each of which is incorporated herein by reference in its entirety.

In several embodiments, the antibodies specifically bind a polypeptide comprising or consisting of a LGR5 polypeptide having at least 60% identity, or at least 70% identity, or at least 80% identity, at least 85% identity, at least 90% identity, at least 95% identity, or at least at least 97% identity, or at least 99% identity, or 100% identity to the human LGR5 polypeptide of NCBI Accession Nos. NP_003658.1 (SEQ ID NO: 47) or fragments thereof. Such fragments can, for example, be at least about 5, 10, 15, 20, 25, 50, 75, 100, 150, 200, 250, 300, 350, 400, 450, 500, 550, 600, 650, 700, 750, 800, 850, or 900 contiguous or non-contiguous amino acids of the LGR5 polypeptide, or any number of contiguous or non-contiguous amino acids in between any of the aforementioned lengths.

In several embodiments, the antibody is antibody 18G7H6A3 and comprises a heavy chain amino acid sequence of SEQ ID NO: 13 and a DNA sequence of SEQ ID NO: 11. In some embodiments, the antibody is antibody 18G7H6A3 and has a heavy chain variable domain comprises SEQ ID NO: 19. In several embodiments, the antibody is antibody 18G7H6A3 and comprises a light chain sequence of SEQ ID NO: 14. In other embodiments, the antibody is antibody 18G7H6A3 and comprises a light chain variable domain of SEQ ID NO: 21.

In some embodiments the antibodies comprise a sequence that is 80%, 81%, 82%, 83%, 84%, 85%, 86%, 87%, 88%, 89%, 90%, 91%, 92%, 93%, 94%, 95%, 96% 97%, 98%, 99%, or 100% identical to the sequence of the above sequences. In some embodiments the antibodies comprise a sequence that is 100% identical to the above antibody sequences over a span of 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 118 residues of the heavy chain, light chain, or variable domains of the above sequences.

In some embodiments the antibodies comprise a sequence that is 80%, 81%, 82%, 83%, 840%, 85%, 86%, 87%, 88%, 89%, 90%, 91%, 92%, 93%, 94%, 95%, 96% 97%, 98%, 99%, or 100% identical to the antibody sequences. In some embodiments the antibodies comprise a sequence that is 84%, 85%, 86%, 87%, 88%, 89%, 90%, 91%, 92%, 93%, 94%, 95%, 96% 97%, 98%, 99%, or 100% identical to the antibody sequences. In some embodiments the antibodies comprise a sequence that is 100% identical to the antibody sequences of over a span of 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, or 111 residues.

In some embodiments, an anti-LGR5 antibody provided herein comprises a heavy chain CDR1 comprising GYSFTAYW (SEQ ID NO:23), a heavy chain CDR2 comprising ILPGSDST (SEQ ID NO:2), and a heavy chain CDR3 comprising ARSGYYGSSQY (SEQ ID NO:3). In some embodiments, an anti-LGR5 antibody provided herein comprises a light chain CDR1 comprising ESVDSYGNSF (SEQ ID NO:4), a light chain CDR2 comprising LTS, and a light chain CDR3 comprising QQNAEDPRT (SEQ ID NO:33).

In some embodiments, an anti-LGR5 antibody provided herein comprises: (a) a heavy chain CDR1 comprising variants of the above sequences having 1, 2, 3, or 4 amino acid substitutions. The antibody may also have a heavy chain CDR2 having a variant comprising 1, 2, 3, or 4 amino acid substitutions. The antibody may also have a heavy chain CDR3 having a variant comprising 1, 2, 3, or 4 amino acid substitutions. In addition to these modifications of the heavy chain, the antibody may also have a light chain CDR1 having a variant comprising 1, 2, 3, or 4 amino acid substitutions. The antibody may also have a light chain CDR2 having a variant comprising 1, 2, 3, or 4 amino acid substitutions. The antibody may also have a light chain CDR3 having 1, 2, 3, or 4 amino acid substitutions. In some embodiments, the amino acid substitutions are conservative amino acid substitutions.

In some embodiments, an anti-LGR5 antibody provided herein comprises an antibody which comprises a heavy chain variable region having at least 80% or 90% sequence identity to the sequences described herein in the attached sequence listing. The antibody may also have a light chain variable region having at least 80% or 90% sequence identity to the antibody sequences described herein.

The percent identity of two amino acid sequences (or two nucleic acid sequences) can be determined, for example, by aligning the sequences for optimal comparison purposes (e.g., gaps can be introduced in the sequence of a first sequence). The amino acids or nucleotides at corresponding positions are then compared, and the percent identity between the two sequences is a function of the number of identical positions shared by the sequences (i.e., % identity=# of identical positions/total # of positions×100). The actual comparison of the two sequences can be accomplished by well-known methods, for example, using a mathematical algorithm. A specific, non-limiting example of such a mathematical algorithm is described in Karlin et al., Proc. Natl. Acad. Sci. USA, 90:5873-5877 (1993), which is incorporated herein by reference in its entirety. Such an algorithm is incorporated into the BLASTN and BLASTX programs (version 2.2) as described in Schaffer et al., Nucleic Acids Res., 29:2994-3005 (2001), which is incorporated herein by reference in its entirety. When utilizing BLAST and Gapped BLAST programs, the default parameters of the respective programs (e.g., BLASTN) can be used. See http://www.ncbi.nlm.nih.gov, as available on Apr. 10, 2002. In one embodiment, the database searched is a non-redundant (NR) database, and parameters for sequence comparison can be set at: no filters; Expect value of 10; Word Size of 3; the Matrix is BLOSUM62; and Gap Costs have an Existence of 11 and an Extension of 1.

Several embodiments also encompass variants of the above described antibodies, including any one of the anti-LGR5 antibodies designated as 18G7Ch. 18G7H6A3 and 18G7H6A1 provided herein, comprising one or more amino acid residue substitutions in the variable light (V_{L}) domain and/or variable heavy (V_{H}) domain. Several also encompass variants of the above described antibodies with one or more additional amino acid residue substitutions in one or more V_{L} CDRs and/or one or more V_{H} CDRs. The antibody generated by introducing substitutions in the V_{H} domain, V_{H} CDRs. V_{L} domain and/or V_{L} CDRs of the above described antibodies can be tested in vitro and in vivo, for example, for its ability to bind to LGR5 (by, e.g., immunoassays including, but not limited to ELISAs and BIAcore).

Various embodiments include antibodies that specifically bind to LGR5 comprising derivatives of the V_{H} domains. V_{H} CDRs, V_{L} domains, or V_{L} CDRs of anti-LGR5 antibodies, such as any one of the anti-LGR5 antibodies designated as 18G7Ch, 18G7H6A3 and 18G7H6A1 provided herein, that specifically bind to LGR5. Standard techniques known to those of skill in the art can be used to introduce mutations (e.g., additions, deletions, and/or substitutions) in the nucleotide sequence encoding an antibody, including, for example, site-directed mutagenesis and PCR-mediated mutagenesis are routinely used to generate amino acid substitutions. In one embodiment, the V_{H} and/or V_{L} CDRs derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original V_{H} and/or V_{L} CDRs. In another embodiment, the V_{H} and/or V_{L} CDRs derivatives have conservative amino acid substitutions (e.g. supra) made at one or more predicted non-essential amino acid residues (i.e., amino acid residues which are not critical for the antibody to specifically bind to LGR5). Alternatively, mutations can be introduced randomly along all or part of the V_{H} and/or V_{L} CDR coding sequence, such as by saturation mutagenesis, and the resultant mutants can be screened for biological activity to identify mutants that retain activity. Following mutagenesis, the encoded antibody can be expressed and the activity of the antibody can be determined.

Several embodiments also encompass antibodies that specifically bind to LGR5 or a fragments thereof, the antibodies comprising an amino acid sequence of a variable heavy chain and/or variable light chain that is at least 45%, at least 50%, at least 55%, at least 60%, at least 65%, at least 70%, at least 75%, at least 80%, at least 85%, at least 90%, at least 95%, or at least 99% identical to the amino acid sequence of the variable heavy chain and/or light chain of any of the antibodies described herein including any one of the anti-LGR5 antibodies including those designated as 18G7Ch, 18G7H6A3 and 18G7H6A1 provided herein.

Another embodiment includes the introduction of conservative amino acid substitutions in any portion of an anti-LGR5 antibody, such as any one of the anti-LGR5 antibodies designated as 18G7Ch. 18G7H6A3 and 18G7H6A1 provided herein. It is well known in the art that “conservative amino acid substitution” refers to amino acid substitutions that substitute functionally-equivalent amino acids. Conservative amino acid changes result in silent changes in the amino acid sequence of the resulting peptide. For example, one or more amino acids of a similar polarity act as functional equivalents and result in a silent alteration within the amino acid sequence of the peptide. Substitutions that are charge neutral and which replace a residue with a smaller residue may also be considered “conservative substitutions” even if the residues are in different groups (e.g., replacement of phenylalanine with the smaller isoleucine). Families of amino acid residues having similar side chains have been defined in the art. Several families of conservative amino acid substitutions are shown in Table 1.

TABLE 1

| Family | Amino Acids |
|---|---|
| non-polar | Trp, Phe, Met, Leu, Ile, Val, Ala, Pro |
| uncharged polar | Gly, Ser, Thr, Asn, Gln, Tyr, Cys |
| acidic/negatively charged | Asp, Glu |
| basic/positively charged | Arg, Lys, His |
| Beta-branched | Thr, Val, Ile |
| residues that influence chain | Gly, Pro |
| orientation | |
| aromatic | Trp, Tyr, Phe, His |

In some embodiments, an anti-LGR5 antibody provided herein binds human LGR5 with a KD of less than about 200 nM, less than about 100 nM, less than about 80 nM, less than about 50 nM, less than about 20 nM, less than about 10 nM, less than about 1 nM, and a range between any of the foregoing values. In some embodiments, an anti-LGR5 antibody provided herein binds LGR5 with an affinity less than about 10 nM, 5 nM, 4 nM, 3 nM, 2 nM, 1 nM, and within a range of any of the foregoing values. In some embodiments, an anti-LGR5 antibody provided herein binds LGR5 with an affinity greater than about 0.0001 nM, 0.001 nM, 0.01 nM, and within a range of any of the foregoing values.

In some embodiments, an anti-LGR5 antibody provided herein binds to an epitope comprising or consisting of or within amino acids T175, E176, Q180, R183, S186, A187, Q189, D247, E248, T251, R254, S257, N258, K260 of SEQ ID NO: 47. In some embodiments, an anti-LGR5 antibody provided herein binds to an epitope comprising or consisting of or within leucine rich repeats 6-9 (See e.g., Chen et al. Genes Dev. 27(12):1345-50 which is incorporated by reference in its entirety). In some embodiments, an anti-LGR5 antibody provided herein binds to an epitope comprising or consisting of or within the convex surface of the LGR5 ecto domain (See e.g., Chen et al. Genes Dev. 27(12):1345-50 which is incorporated by reference in its entirety).

### Certain Humanized Anti-LGR5 Antibodies

Some embodiments of the methods and compositions provided herein include the use of certain anti-LGR5 antibodies or antigen binding fragments thereof, including those derived from murine antibody ‘18G7.1’. Human germline sequences were used as the acceptor frameworks for humanizing the murine antibody 18G7.1. To find the closest germline sequences, the most similar expressed light chain and the most similar heavy chain were identified in a database of germline sequences by NCI IgBLAST (ncbi.nlm.nih.gov/igblast/). In this search the CDR sequences of 18G7.1 were masked. The selection of the most suitable expressed sequence included checking for sequence identity of the canonical and interface residues, and checking for the similarity in CDR loop lengths.

In order to identify potential structural conflicts in key structural framework residues between the candidate humanized sequence and the parent murine monoclonal antibody 18G7.1, a three-dimensional model was generated. A composite of antibody structures was used to create a homology model with grafted candidate humanized sequences followed by molecular energy minimization. Structural analysis using computer software Pymol, was used to identify residues that could potentially negatively impact proper folding.

From this analysis, six candidate VH chains were constructed that included: 1) a functional human framework containing selected substitutions within the candidate humanized framework region based on analysis of likely impact on folding and ii) the parental 18G7.1 murine antibody CDRs (SEQ ID NOs: 1, 2, and 3), fused in-frame to the human IgG1 constant region are chemically synthesized.

Similarly, two candidate VL chains were constructed that included: 1) a functional human framework containing selected substitutions within the candidate humanized framework region based on analysis of likely impact on folding and ii) the parental 18G7.1 murine antibody CDRs (SEQ ID NOs: 4, 5, and 6). The candidate VL chain and the candidate VH chain fused in-frame to the human IgG1 constant region were chemically synthesized.

Selected candidate variant humanized heavy and light chain combinations were tested for functionality by co-transfection into mammalian cells. Each of the six candidate humanized 18G7.1 heavy chains described above were co-transfected with one of the candidate 18G7.1 light chains into HEK 293 cells, and conditioned media was assayed for LGR5 antigen binding activity by flow cytometry. In addition, three candidate humanized 18G7.1 heavy chains described above were co-transfected with the second candidate 18G7.1 light chain into HEK 293 cells, and conditioned media was assayed for LGR5 antigen binding activity by flow cytometry. The 18G7.1 candidate heavy chain/light chain combination (humanization variant) known as 18G7H6, and which exhibited the most robust binding was selected for affinity maturation.

In order to increase the affinity of the selected humanized variant 18G7H6, a combination of alanine scanning mutagenesis and saturation mutagenesis was employed. Residues in heavy chain CDR1 and light chain CDR1 and CDR3 were mutated to alanine, transfected into HEK 293 cells, and the resultant conditioned media was assayed for LGR5 antigen binding activity by flow cytometry. Saturation mutagenesis was performed on heavy chain CDR3, in which every residue in CDR3 was mutated to each of the 19 naturally occurring amino acids except the original amino acid identity at that position. Each of the mutants were transfected into HEK 293 cells, and the resultant conditioned media was assayed for LGR5 antigen binding activity by flow cytometry.

These mutations were incorporated at increasing number into 3 constructs. These three constructs were then transfected into HEK 293 cells, and the resultant conditioned media was assayed for LGR5 antigen binding activity by flow cytometry. Two constructs 18G7H6A1 and 18G7H6A3 (also known as BNC101) were selected for further characterization. TABLE 2 lists certain sequences of the antibodies.

TABLE 2

| Description | SEQ ID NO: |
|---|---|
| 18G7.1 Heavy Chain CDR1 Amino Acid | 1 |
| 18G7.1 Heavy Chain CDR2 Amino Acid | 2 |
| 18G7.1 Heavy Chain CDR3 Amino Acid | 3 |
| 18G7.1 Light Chain CDR1 Amino Acid | 4 |
| 18G7.1 Light Chain CDR2 Amino Acid | 5 |
| 18G7.1 Light Chain CDR3 Amino Acid | 6 |
| 18G7H6A1 Heavy Chain DNA | 7 |
| 18G7H6A1 Light Chain DNA | 8 |
| 18G7H6A1 Heavy Chain Amino Acid | 9 |
| 18G7H6A1 Light Chain Amino Acid | 10 |
| 18G7H6A3 Heavy Chain DNA | 11 |
| 18G7H6A3 Light Chain DNA | 12 |
| 18G7H6A3 Heavy Chain Amino Acid | 13 |
| 18G7H6A3 Light Chain Amino Acid | 14 |
| 18G7Ch Heavy Chain DNA | 15 |
| 18G7Ch Light Chain DNA | 16 |
| 18G7Ch Heavy Chain Amino Acid | 17 |
| 18G7ch Light Chain Amino Acid | 18 |
| 18G7H6A3 Heavy Chain Variable Domain Amino Acid | 19 |
| 18G7H6A3 Heavy Chain Variable Domain DNA | 20 |
| 18G7H6A3 Light Chain Variable Domain | 21 |
| 18G7H6A3 Light Chain Variable Domain DNA | 22 |
| 18G7H6A3 Heavy Chain CDR1 Amino Acid | 23 |
| 18G7H6A3 Heavy Chain CDR1 DNA | 24 |
| 18G7H6A3 Heavy Chain CDR2 Amino Acid | 25 |
| 18G7H6A3 Heavy Chain CDR2 DNA | 26 |
| 18G7H6A3 Heavy Chain CDR3 Amino Acid | 27 |
| 18G7H6A3 Heavy Chain CDR3 DNA | 28 |
| 18G7H6A3 Light Chain CDR1 Amino Acid | 29 |
| 18G7H6A3 Light Chain CDR1 DNA | 30 |
| 18G7H6A3 Light Chain CDR2 Amino Acid | 31 |
| 18G7H6A3 Light Chain CDR2 DNA | 32 |
| 18G7H6A3 Light Chain CDR3 Amino Acid | 33 |
| 18G7H6A3 Light Chain CDR3 DNA | 34 |
| 18G7H6A1 Heavy Chain CDR1 Amino Acid | 35 |
| 18G7H6A1 Heavy Chain CDR1 DNA | 36 |
| 18G7H6A1 Heavy Chain CDR2 Amino Acid | 37 |
| 18G7H6A1 Heavy Chain CDR2 DNA | 38 |
| 18G7H6A1 Heavy Chain CDR3 Amino Acid | 39 |
| 18G7H6A1 Heavy Chain CDR3 DNA | 40 |
| 18G7H6A1 Light Chain CDR1 Amino Acid | 41 |
| 18G7H6A1 Light Chain CDR1 DNA | 42 |
| 18G7H6A1 Light Chain CDR2 Amino Acid | 43 |
| 18G7H6A1 Light Chain CDR2 DNA | 44 |
| 18G7H6A1 Light Chain CDR3 Amino Acid | 45 |
| 18G7H6A1 Light Chain CDR3 DNA | 46 |
| LGR5 Amino Acid Sequence | 47 |
| 18G7H6A1 Heavy Chain Variable Amino acid | 48 |
| 18G7H6A1 Light Chain Variable Amino acid | 49 |

Certain characteristics of anti-LGR5 antibodies, such as 18G7H6A3, are disclosed in U.S. Pat. No. 9,546,214 which is expressly incorporated by reference in its entirety. Certain characteristics are summarized below.

### Characteristics of Certain Anti-LGR5 Antibodies

In a FACS-based assay using chinese hamster ovary (CHO) expressing a recombinant LGR5, 18G7H6A1 and 18G7H6A3 were each determined to have an EC50<10 nM for human LGR5 binding. In another study, 18G7H6A3 was found to strongly bind human and cyno LGR5, but not bind to rat or mouse LGR5. In an ELISA-based plate binding assay, 18G7H6A3 was determined to bind to an LGR5 ectodomain-IgG-Fc fiusion protein with an EC50 of 300 pM.

Cell surface expression levels of LGR5 were determined for various human cell lines using flow cytometry. CT1 colorectal tumor cells and pancreatic cancer cell lines Panc-1, Capan2 and CFPAC were among the highest LGR5 expressors. Moderate expression levels were observed in pancreatic cancer cell lines (AsPC-1, SW1990, HPAFII), cisplatin-resistant ovarian cancer cell lines (OVCAR8/CP, A2780/CP and Igrov1/CP) as well as colon, breast and ovarian cancer cell lines (SW48, Hs578T and OVCAR3). Low but detectable levels of LGR5 cell surface expression were observed in colon (SW480, LoVo) and breast cancer cell lines (MDA-MB-231). TABLE 3 summarizes the data for 18G7H6A3 FACS binding to various tumor cell lines.

TABLE 3

| Tumor Cell line | 18G7H6A3 (18G7.1) | IgG |
|---|---|---|
| CT1 | + | − |
| CT3 | + | − |
| DLD1 | +/− | − |
| Ls174T | +/− | − |
| LoVo | +/− | − |
| SW48 | + | − |
| SW480 | +/− | − |
| SW620 | +/− | − |
| HCT116 | +/− | − |
| Breast | | |
| MDA-MB-231 | +/− | − |
| MDA-MB-231 LM2 | +/− | − |
| Hs578T | + | − |
| CN34 | +/− | − |
| CN34 LM1 | +/− | − |
| Prostate | | |
| PC-3 | +/− | − |
| PCSD1 | +/− | − |
| Ovarian | | |
| OVCAR-3 | + | − |
| SK-OV-3 | +/− | − |
| SK-OV-3/CP | +/− | − |
| OVCAR8/CP | + | − |
| Igrov1/CP | + | − |
| A2780/CP | + | − |
| Lung | | |
| HOP-62 | +/− | − |
| Pancreatic | | |
| AsPC-1 | + | − |
| Capan2 | ++ | − |
| HPAFII | + | − |
| Sw1990* | + | − |
| CFPAC | ++ | − |
| PANC-1 | ++ | − |

Internalization of 18G7H6A3 was examined on CHO cell overexpressing LGR5. Cells were stained with 100 nM antibody for 30 min-2 hrs at 4° C., excess Ab was washed off and stained cells were incubated at either 4° C. or 37° C. Cells were stained with AlexaFluor488-conjugated secondary antibodies at various time points to monitor internalization of cell surface-bound antibodies. Upon incubation at 37° C., the internalized rate had a measured t1/2 value for surface localization of 6.703±1.282 minutes. Internalization was largely blocked by incubation at 4° C. although some decrease in surface-bound antibody was observed.

An epitope mapping experiment was performed using hydrogen deuterium exchange mass spectrometry to characterize the specific region(s) of LGR5 that antibody 18G7H6A3 binds. Hydrogen/deuterium (H/D)-exchange data indicated that 18G7H6A3 binds to amino acids T175, E176, Q180, R183, S186, A187, Q189, D247, E248, T251, R254, S257, N258, K260 of SEQ ID NO: 47 within the convex surface of leucine rich repeats 6-9, on the opposite of the face of the R-spondin binding site as identified by X-ray crystallographic studies. (See e.g., Chen et al. Genes Dev. 27(12):1345-50 which is incorporated by reference in its entirety). These data show that the residues involved in binding of LGR5 to the R-spondins are not involved in binding 18G7H6A3. These preliminary results do not preclude that fact that other structural elements in LGR5 may be involved in the binding site of 18G7H6A3.

### In Vivo Activities of Certain Anti-LGR5 Antibodies

In a xenograft model using human colon CT1 cells derived from a patient with stage IV metastatic colon cancer transplanted into mice, 18G7H6A3 showed significant anti-tumor activity in vivo compared to PBS and MOPC antibody controls during the course of 4 doses (15 mg/kg, twice weekly). While antibody 18G7H6A1 showed anti-tumor activity, monoclonal 18G7H6A3 showed superior activity to both 18G7H6A1 and the parental murine chimeric 18G7Ch antibody. TABLE 4 shows percent CT1 tumor volume reduction (group vs MOPC) after 1-4 doses of Lgr5+ Abs.

TABLE 4

# of Doses:

| | 1 | 2 | 3 | 4 |
|---|---|---|---|---|
| 18G7Ch | 9.2% | 30.6% | 19.5% | 29.0% |
| 18G7H6A1 | 17.5% | 19.1% | 14.2% | 19.0% |
| 18G7H6A3 | 38.8% | 42.0% | 28.9% | 35.4% |

In a xenograft model using human colon CT3 cells derived from a patient with stage IV metastatic colon cancer transplanted into mice, 18G7H6A1 showed anti-tumor activity, and 18G7H6A3 showed significant anti-tumor activity compared to PBS and MOPC antibody controls after 4 doses (15 mg/kg, twice weekly). 18G7H6A3 showed superior activity to the parental murine chimeric 18G7Ch antibody and equivalent activity to 18G7H6A1. TABLE 5 shows percent CT3 tumor volume reduction (group vs MOPC) after n dose of test Abs.

TABLE 5

# of Ab Doses:

| | 1 | 2 | 3 | 4 |
|---|---|---|---|---|
| 18G7Ch | 22.6% | 8.9% | 17.0% | 13.8% |
| 18G7H6A1 | 18.3% | 12.6% | 28.8% | 28.7% |
| 18G7H6A3 | 34.2% | 38.1% | 23.4% | 28.2% |

In a xenograft model using human CT3 cells grown under CSC conditions, treated with 18G7H6A3 in combination with a FOLFIRI regimen, analyses of tumor volume showed that administration of 18G7H6A3 and FOLFIRI further reduced growth of CT3 tumors compared to FOLFIRI only. Cells isolated from mice treated with 18G7H6A3 in combination with FOLFIRI were replanted into mice. Transplanted cells had greatly decreased tumorigenicity as compared to cells isolated from mice treated with FOLFIRI alone. In addition, the re-implanted cells from the 18G7H6A3 FOLFIRI combination had a significantly slower tumor growth profile and a delayed time to progression compared to FOLFIRI alone. These data indicate that 18G7H6A3 in combination with FOLFIRI effectively targets the tumor initiating or cancer stem cell population.

In a xenograft model using human pancreatic AsPC-1 cells, 18G7H6A3 as single agent inhibited tumor growth in mice compared to saline and/or control IgG up to nearly 40% at day 41 post implantation. In addition, a combination of 18G7H6A3 and gemcitabine significantly inhibited tumor growth in the AsPC-1 model (up to 36% at day 61 post implantation) compared to gemcitabine alone. 18G7H6A3 as single agent also provided some inhibition in tumor growth compared to PBS and control IgG up to day 65.

In a xenograft model using human breast MDA-MB-231-LM3 cells, 18G7H6A3 showed significant anti-tumor activity in mice compared to PBS (60.7% tumor growth inhibition) or MOPC antibody (49.3% tumor growth inhibition) controls. Cells isolated from mice treated with 18G7H6A3 in combination with paclitaxel were transplanted into mice. Such cells had greatly decreased tumorigenicity as compared to cells isolated from mice treated with paclitaxel alone. In addition, the re-implanted cells from the 18G7H6A3 plus paclitaxel tumors had a significantly slower tumor growth profile and a delayed time to progress compared to paclitaxel alone. These data indicate that 18G7H6A3 in combination with paclitaxel effectively targets the tumor initiating or cancer stem cell population.

In a xenograft model using human pancreatic PANC1cells, 18G7H6A3 alone inhibited tumor growth in mice (up to 30% at day 70 post implantation), 18G7H6A3 in combination with gemcitabine significantly inhibited tumor growth (up to 52% at day 80 post implantation) compared to gemcitabine alone group. Cells isolated from mice treated with 18G7H6A3 in combination with gemcitabine were transplanted into mice. Such cells had greatly decreased tumorigenicity compared to cells isolated from mice treated with gemcitabine alone. Re-implanted PANC1 tumors treated with combination of gemcitabine and 18G7H6A3 showed reduction in the frequency of engraftment in mice implanted with 4500 cells (40% in gemcitabine vs. 20% in combination) and also in mice implanted with 13500 cells (100% in gemcitabine vs. 70% in combination). These data indicate that 18G7H6A3 in combination with gemcitabine effectively targets the tumor initiating or cancer stem cell population.

In a xenograft model using human pancreatic JH109 cells, 18G7H6A3 treatment alone did not affect tumor growth in mice. 18G7H6A3 in combination with Nab-paclitaxel and gemcitabine chemotherapy led to a significantly greater degree of tumor inhibition compared to chemotherapy alone. 18G7H6A3 combined with chemotherapy led to 77% greater tumor growth inhibition compared to chemotherapy alone. Three mice treated with the 18G7H7A3 chemotherapy combination had complete eradication of their tumor.

In a xenograft model using human BMCRC086 cells, 18G7H6A3 in combination with FOLFIRI showed significant anti-tumor activity in mice compared to FOLFIRI alone.

In a xenograft model using human small cell lung cancer cells derived from BLG293 tumors, 18G7H6A3 showed significant anti-tumor activity in mice compared to PBS (24.9% tumor growth inhibition) and MOPC antibody (24.7% tumor growth inhibition) controls.

### Antibody Drug Conjugates

Embodiments of the methods and compositions provided herein include ADCs. In some such embodiments an antibody or antigen binding fragment thereof that specifically binds to human LGR5 is conjugated to a therapeutic agent, such as a drug. In some embodiments, the therapeutic agent comprises a cytostatic or cytotoxic agent. In some embodiments, the therapeutic agent is a DNA damaging agent, or a microtubule inhibitor. Examples of therapeutic agents include dolastatins and auristatins, including monomethyl auristatin E (MMAE) and monomethyl auristatin F (MMAF), amanitins such as α-amanitin, β-amanitin, or γ-amanitin, DNA minor groove binding agents such as duocarmycin derivatives, alkylating agents such as modified or dimeric pyrrolobenzodiazepines (PBD), mechlorethamine, thioepa, chlorambucil, melphalan, carmustine, lomustine, cyclothosphamide, busulfan, dibromomannitol, streptozotocin, mitomycin C and cisdichlorodiamine platinum (11) (DDP) cisplatin, splicing inhibitors such as meayamycin analogs or derivatives, tubular binding agents such as epothilone analogs and paclitaxel and DNA damaging agents such as calicheamicins and esperamicins, antimetabolites such as methotrexate, 6-mercaptopurine, 6-thioguanine, cytarabine, and 5-fluorouracil decarbazine, anti-mitotic agents such as vinblastine and vincristine and anthracyclines such as daunorubicin (also known as daunomycin) and doxorubicin and pharmaceutically acceptable salts or solvates, acids or derivatives thereof. In some embodiments, therapeutic agents include antimitotic agents such as allocolchicine; auristatins, such as MMAE and MMAF; halichondrin B; cemadotin; colchicine; cholchicine derivative (N-benzoyl-deacetyl benzamide); dolastatin-10; dolastatin-15; maytansine; maytansinoids, such as DM1; rhozoxin; paclitaxel; paclitaxel derivative ((2′-N-[3-(dimethylamino)propyl]glutaramate paclitaxel); docetaxel; thiocolchicine; trityl cysteine; vinblastine sulfate; and vincristine sulfate. In some embodiments, therapeutic agents include alkylating antineoplastic agents such as Carboquone: Carmustine; Chlornaphazine; Chlorozotocin; Duocarmycin: Evofosfamide; Fotemustine; Glufosfamide; Lomustine; Mannosulfan; Nimustine; Phenanthriplatin; Pipobroman; Ranimustine: Semustine; Streptozotocin; ThioTEPA; Treosulfan; Triaziquone; Triethylenemelamine; and Triplatin tetranitrate. In some embodiments, the therapeutic agent can include MMAF, MMAE, monomethyl dolastatin 10, duocarmycin, maytansanoid 1, dualstatin 3, calicheamicin, and duocamycin. In some embodiments, the therapeutic agent is a DNA damaging agent, or a microtubule inhibitor. Examples of microtubule inhibitors include Cabazitaxel, Colcemid, Colchicine, Cryptophycin, Cytoskeletal drugs, Demecolcine, Docetaxel, 2-Methoxyestradiol, Nocodazole, Paclitaxel, Taccalonolide, Taxane, and Vinblastine. More examples of therapeutic agents useful with the methods and compositions provided herein are disclosed in U.S. 2017/0137533; U.S. 2017/0158769; U.S. 2017/0151344; and U.S. 2017/0136130 which are each incorporated herein by reference in its entirety.

In some such embodiments an antibody or antigen binding fragment thereof that specifically binds to human LGR5 is conjugated to a therapeutic agent via a linker. In some embodiments, a linker may be polyvalent such that it covalently links more than one agent to a single site on an antibody or antigen binding fragment thereof, or monovalent such that it covalently links a single agent to a single site on an antibody or antigen binding fragment thereof. Example linkers useful to link therapeutic agents and the antibodies or antigen binding fragments thereof provided herein are described in U.S. Pat. Nos. 7,223,837; 8,568,728; 8,535,678; WO 2009/073445; WO 2010/068795; WO 2010/138719; WO 2011/120053; WO 2011/171020; WO 2013/096901; WO 2014/008375; WO 2014/093379; WO 2014/093394; and WO 2014/093640, each of which is incorporated herein by reference in its entirety.

In some embodiments, a linker can be a cleavable linker. Cleavable linkers may include chemically or enzymatically unstable or degradable linkages. Cleavable linkers generally rely on processes inside the cell to liberate the therapeutic agent, such as reduction in the cytoplasm, exposure to acidic conditions in the lysosome, or cleavage by specific proteases or other enzymes within the cell. Cleavable linkers generally incorporate one or more chemical bonds that are either chemically or enzymatically cleavable while the remainder of the linker is noncleavable. In certain embodiments, a linker comprises a chemically labile group such as hydrazone and/or disulfide groups. Linkers comprising chemically labile groups exploit differential properties between the plasma and some cytoplasmic compartments. The intracellular conditions to facilitate therapeutic agent release for hydrazone containing linkers are the acidic environment of endosomes and lysosomes, while the disulfide containing linkers are reduced in the cytosol, which contains high thiol concentrations, e.g., glutathione. In certain embodiments, the plasma stability of a linker comprising a chemically labile group may be increased by introducing steric hindrance using substituents near the chemically labile group.

In some embodiments, a linker can be a non-cleavable linker. For non-cleavable linkers, the release of therapeutic agent may not depend on the differential properties between the plasma and some cytoplasmic compartments. The release of the therapeutic agent is postulated to occur after internalization of the ADC via antigen-mediated endocytosis and delivery to lysosomal compartment, where the antibody is degraded to the level of amino acids through intracellular proteolytic degradation. This process releases a drug derivative, which is formed by the therapeutic agent, the linker, and the amino acid residue to which the linker was covalently attached. The amino acid drug metabolites from conjugates with non-cleavable linkers are more hydrophilic and generally less membrane permeable, which leads to less bystander effects and less nonspecific toxicities compared to conjugates with a cleavable linker. In general, ADCs with non-cleavable linkers have greater stability in circulation than ADCs with cleavable linkers. Non-cleavable linkers may be alkylene chains, or may be polymeric in nature, such as, for example, those based upon polyalkylene glycol polymers, amide polymers, or may include segments of alkylene chains, polyalkylene glycols and/or amide polymers. Example cleavable and non-cleavable linkers useful to link therapeutic agents and the antibodies or antigen binding fragments thereof provided herein are described in U.S. 2017/0151344 which is incorporated herein by reference in its entirety. Examples methods of methods to link drugs to antibodies or antigen binding fragments thereof are disclosed in Behrens C. R. et al., MAbs. 2014 Jan. 1; 6(1): 46-53; and Zhou q., et al, Anticancer Agents Med Chem. 2015; 15(7):828-36, which are each incorporated by reference in its entirety.

Some embodiments of the methods and compositions provided herein include preparing an ADC. Some such embodiments can include linking a linker to a therapeutic agent, and the linked therapeutic agent can be conjugated to an antibody or antigen binding fragment thereof via the linker. Some such embodiments can include linking a linker to an, and the linked antibody or antigen binding fragment thereof can be conjugated to a therapeutic agent via the linker. Some embodiments also include purifying the ADC.

### Methods of Treatment

Some embodiments of the methods and compositions provided herein include treating a subject having a cancer. As used herein “treating” or “treatment” or “to treat” can refer to both therapeutic effects that cure, slow down, lessen symptoms of, and/or halt progression of a diagnosed pathologic condition or disorder, such as a cancer, and prophylactic measures that prevent and/or slow the development of a targeted pathologic condition or disorder, such as a cancer. Some such embodiments include administering an effective amount of an ADC provided herein to a subject in need thereof. In some embodiments, the cancer comprises a solid tumor. In some embodiments, the cancer can include lung cancer, breast cancer, colon cancer, and pancreatic cancer. In some embodiments, the cancer can include a cell such as a triple negative breast cancer cell, a colon cancer cell having a mutation in a gene selected from the group consisting of K-Ras, H-Ras, APC, PI3K, PTEN, STK11, RB1, TP53, FGFR2, VANGL2, and ISCO, and a small cell lung cancer cell. In some embodiments, the cancer can include a cancer stem cell. In some embodiments, the subject is mammalian, such as human.

Some embodiments also include administering an additional therapy in combination with the ADC provided herein. In some embodiments, the additional therapy can include radiotherapy, and a chemotherapeutic agent. In some embodiments, administration of the ADC is concurrent with administration of the additional therapy. In some embodiments, the chemotherapeutic agent can include folinic acid, fluorouracil, irinotecan, gemcitabine, paclitaxel, nab-paclitaxel, ERBITUX (cetuximab), PI3K/mTOR dual inhibitor (NVP), and SN38. In some embodiments, the additional therapy comprises folinic acid, fluorouracil, and irinotecan. More examples of chemotherapeutic agents useful with the methods and compositions provided herein are disclosed in U.S. 2017/0137533 which is incorporated herein by reference in its entirety.

Some embodiments of the methods and compositions provided herein include inhibiting growth of a neoplastic cell. Some such embodiments include contacting the cell with an ADC provided herein. In some embodiments, the neoplastic cell can include lung cancer cell, breast cancer cell, colon cancer cell, and pancreatic cancer cell. In some embodiments, the neoplastic cell can include a cell such as a triple negative breast cancer cell, a colon cancer cell having a mutation in a gene selected from the group consisting of K-Ras, H-Ras, APC, PI3K. PTEN, STK11, RB1, TP53, FGFR2, VANGL2, and ISCO, and a small cell lung cancer cell. In some embodiments, the neoplastic cell can include a cancer stem cell. In some embodiments, the neoplastic cell is mammalian, such as human. In some embodiments, the neoplastic cell is in vitro. In some embodiments, the neoplastic cell is in vivo.

In some embodiments provided herein, ADCs can be formulated in various ways using art recognized techniques. In some embodiments, therapeutic compositions provided herein can be administered neat or with a minimum of additional components while others may optionally be formulated to contain suitable pharmaceutically acceptable carriers. As used herein, “pharmaceutically acceptable carriers” comprise excipients, vehicles, adjuvants and diluents that are well known in the art and can be available from commercial sources for use in pharmaceutical preparation (see, e.g., Gennaro (2003) Remington: The Science and Practice of Pharmacy with Facts and Comparisons: Drugfacts Plus. 20th ed., Mack Publishing; Ansel et al. (2004) Pharmaceutical Dosage Forms and Drug Delivery Systems, 7.sup.th ed., Lippencott Williams and Wilkins; Kibbe et al. (2000) Handbook of Pharmaceutical Excipients, 3.sup.rd ed., Pharmaceutical Press.).

Some embodiments of the methods and compositions provided herein include formulations of an ADC suitable for parenteral administration (e.g., by injection), and can include aqueous or non-aqueous, isotonic, pyrogen-free, sterile liquids (e.g., solutions, suspensions), in which the active ingredient is dissolved, suspended, or otherwise provided (e.g., in a liposome or other microparticulate).

The particular dosage regimen of certain embodiments, i.e., dose, timing and repetition, can depend on the particular subject, as well as empirical considerations such as pharmacokinetics (e.g., half-life, clearance rate, etc.). Determination of the frequency of administration may be made by persons skilled in the art, such as an attending physician based on considerations of the condition and severity of the condition being treated, age and general state of health of the subject being treated and the like. Frequency of administration may be adjusted over the course of therapy based on assessment of the efficacy of the selected composition and the dosing regimen. Such assessment can be made on the basis of markers of the specific disease, disorder or condition. In embodiments where the individual has cancer, these include direct measurements of tumor size via palpation or visual observation: indirect measurement of tumor size by x-ray or other imaging techniques; an improvement as assessed by direct tumor biopsy and microscopic examination of a tumor sample: the measurement of a surrogate biomarker or an antigen identified according to the methods described herein; reduction in the number of proliferative or tumorigenic cells, maintenance of the reduction of such neoplastic cells; reduction of the proliferation of neoplastic cells; or delay in the development of metastasis.

### Kits

Some embodiments of the methods and compositions provided herein include kits. In some embodiments, a kit can include an ADC provided herein. In some embodiments, the ADC is lyophilized. In some embodiments, the ADC is in aqueous solution. In some embodiments, the kit includes a pharmaceutical carrier for administration of the ADC. In some embodiments, the kit also includes a chemotherapeutic agent. In some embodiments, the chemotherapeutic agent is selected from folinic acid, fluorouracil, irinotecan, gemcitabine and Abraxane. In some embodiments, the kit include components to maintain the activity of the ADC, such as a cooling agent, such as ice or dry ice.

### EXAMPLES

### Example 1-Proliferation Assay with Conjugated Secondary Antibodies

Chinese hamster ovary cells (CHO), and CHO cells expressing human LGR5 (CHO-LGR5) were treated with a primary human anti-LGR5 antibody (C12) and a secondary anti-human antibody drug conjugate (ADC) that would bind to the primary antibody. Antibody C12 is disclosed in U.S. Pat. No. 9,221,906 which is incorporated by reference in its entirety. Cells were plated at 5000 cells/well in a 96-well plate in F12 media+10% fetal bovine serum÷antibiotic/antimitotic to form a monolayer. ADCs (Moradec, San Diego Calif.) are listed in TABLE 6.

TABLE 6

| Secondary ADC | Conjugated drug | Linker |
|---|---|---|
| NC-MMAF | monomethyl auristatin F (MMAF) | non-cleavable |
| CL-MD10 | monomethyl dolastatin 10 (MD10) | cleavable |
| CL-MMAE | monomethyl auristatin E (MMAE) | cleavable |
| CL-DMSA | duocarmycin (DMSA) | cleavable |
| NC-DM1 | maytansanoid 1 (DM1) | non-cleavable |
| CK-DUA3 | dualstatin 3 (DUA3) | lysine cleavable |
| CK-CAL | calicheamicin (CAL) | lysine cleavable |

TABLE 7 lists concentrations of primary antibody and secondary ADC used to treat cells. Cells treated with CK-CAL were not assessed at 30 nM C12, and cells treated with CK were not assessed at 30 pM C12.

TABLE 7

| Primary antibody C12 | | Secondary ADC | |
|---|---|---|---|
| 0 | | 0 | |
| 30 | pM | 150 | pM |
| 100 | pM | 500 | pM |
| 300 | pM | 1.5 | nM |
| 1 | nM | 5 | nM |
| 3 | nM | 15 | nM |
| 10 | nM | 50 | nM |
| 30 | nM | 150 | nM |

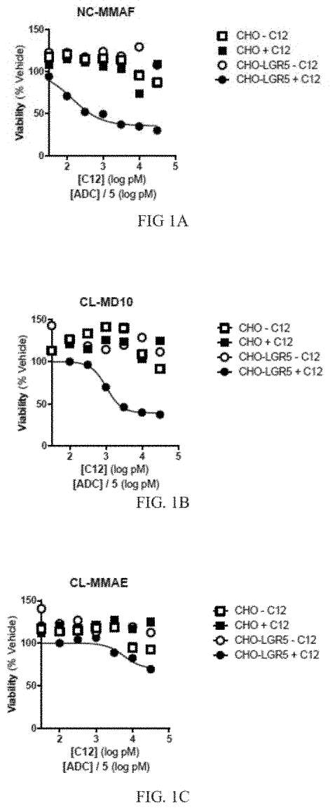

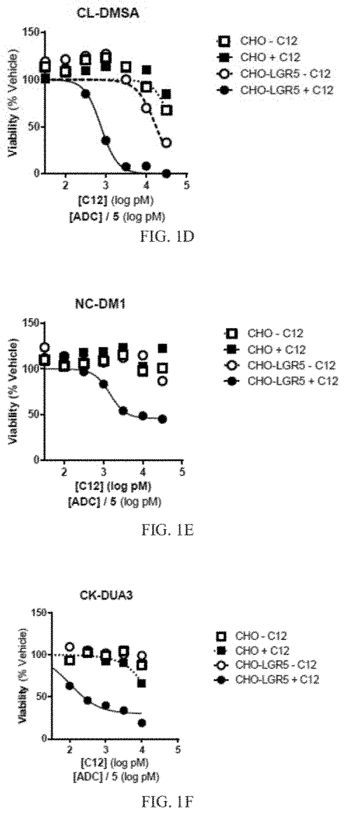

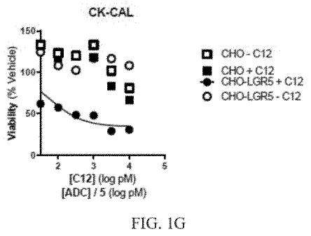

Cell viability was determined using an MTS proliferation assay after three days of treatment. Briefly, 20 μl MTS reagent which included a tetrazolium compound [3-(4,5-dimethylthiazol-2-yl)-5-(3-carboxymethoxyphenyl)-2-(4-sulfophenyl)-2H-tetrazolium, inner salt; MTS] and an electron coupling reagent (phenazine ethosulfate; PES) was added to cells grown in 96 well plates to a total volume of 100 μl. Plates were incubated for 1-4 hours at 37° C., and the absorbance of the cell medium was measured at 490 nm. The quantity of formazan product as measured by the amount of 490 nm absorbance was directly proportional to the number of living cells in culture. IC50 values were also determined. Results are depicted in FIGS. 1A-1G, and in TABLE 8.

TABLE 8

| Secondary ADC | IC50 (nM) | Curve min (%) |
|---|---|---|
| NL-NLMAF | 0.140 | 35.8 |
| CL-MD10 | 1.04 | 39.4 |
| CL-MMAE | 5.81 | 69.9 |
| CL-DMSA | 0.765 | 0 |
| NC-DM1 | 1.45 | 46.2 |
| CK-DUA3 | 0.159 | 49.7 |
| CK-CAL | 0.060 | 34.4 |

As shown, the combination of the C12 antibody and the lysine cleavable calicheamicin (CK-CAL) antibody had the lowest IC50 score of 0.060. The anti-human antibody that was non-cleavably bound to monomethyl auristatin F (NL-MMAF) had the next lowest IC50 value of 0.140.

### Example 2-Proliferation Assay with Conjugated Secondary Antibodies

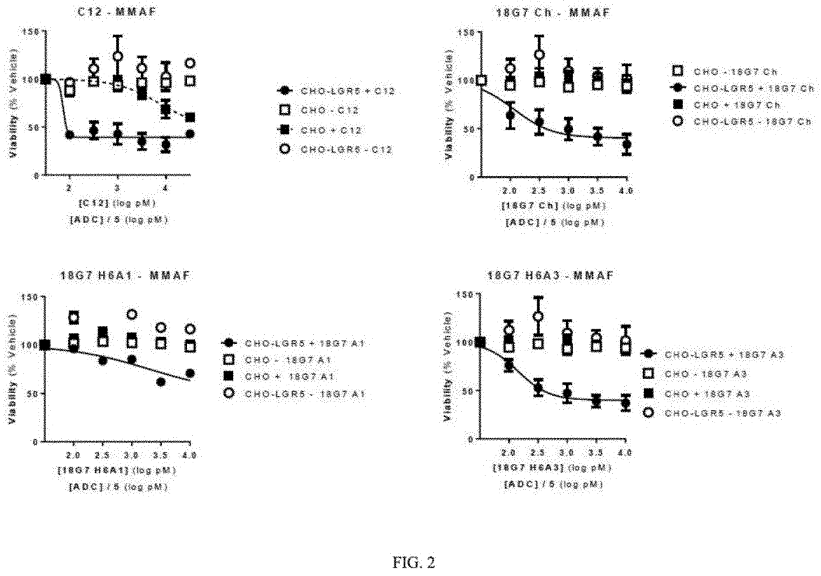

CHO and CHO-LGR5 were treated with various primary human anti-LGR5 antibodies and the secondary anti-human ADC (NL-MMAF) for three days. Cell viability was measured using an MTS assay as described in Example 1. Primary antibodies included: C12, 18G7Ch, 18G7H6A1, and 18G7H6A3. The concentrations of the primary antibodies and the secondary ADC used to treat the cells are listed in TABLE 9. Results are shown in FIG. 2, and TABLE 10.

TABLE 9

| Primary antibody | | Secondary ADC | |
|---|---|---|---|
| 0 | | 0 | |
| 30 | pM | 150 | pM |
| 100 | pM | 500 | pM |
| 300 | pM | 1.5 | nM |
| 1 | nM | 5 | nM |
| 3 | nM | 15 | nM |
| 10 | nM | 50 | nM |
| 30 | nM* | 150 | nM* |

*C12 experiment only

TABLE 10

| | | 95% conf. interval |
|---|---|---|
| Treatment | IC50 (nM) | of IC50% value (nM) |
| ADC alone | N/A | N/A |
| C12 + ADC (NL-MMAF) | 0.073 | Wide |
| 18G7Ch + ADC (NL-MMAF) | 0.115 | 0.041-0.315 |
| 18G7H6A3 + ADC (NL-MMAF) | 0.142 | 0.079-0.254 |

Each of the four tested primary antibodies, with the secondary anti-human ADC (NL-MMAF) was effective to reduce cell viability of CHO expressing human LGR5.

### Example 3-Tumorsphere Assay with Conjugated Secondary Antibodies in Human Cells

Tumorspheres are solid, spherical formations that develop from the proliferation of single cancer stem/progenitor cells. Tumorspheres can be induced to develop from a population of cells by growing the cells in serum-free, and/or non-adherent conditions. Tumorspheres can therefore be indicative of the number of cancer stem cells in a population of cells. Human CT3 cells are low passage primary cells derived from a patient with stage IV metastatic colon cancer that can be induced to form tumorspheres, and include mutations in the following genes: K-Ras, H-Ras, APC, PI3K. PTEN, STK11, RB1, TP53, FGFR2, VANGL2, and ISCO.

Human CT3 cells which express LGR5 were grown in culture under normal conditions, harvested using Accutase cell dissociation reagent and a single cell suspension formed by passing cells through a cell strainer. The single cell suspension was plated at 500 cells/well into 96-well low attachment plates in specific cancer stem cell (CSC) media. The cells were treated with various primary human anti-LGR5 antibodies and a secondary anti-human ADC (CL-DMSA) at a 1:1 ratio of primary and secondary antibodies at the following concentrations: 0.03, 0.1, 0.3, 1, 3, and 10 nM. Primary antibodies included: C12, 18G7Ch, 18G7H6A1, and 18G7H6A3. The treated cells were incubated for 7 days and the number of tumorspheres were counted.

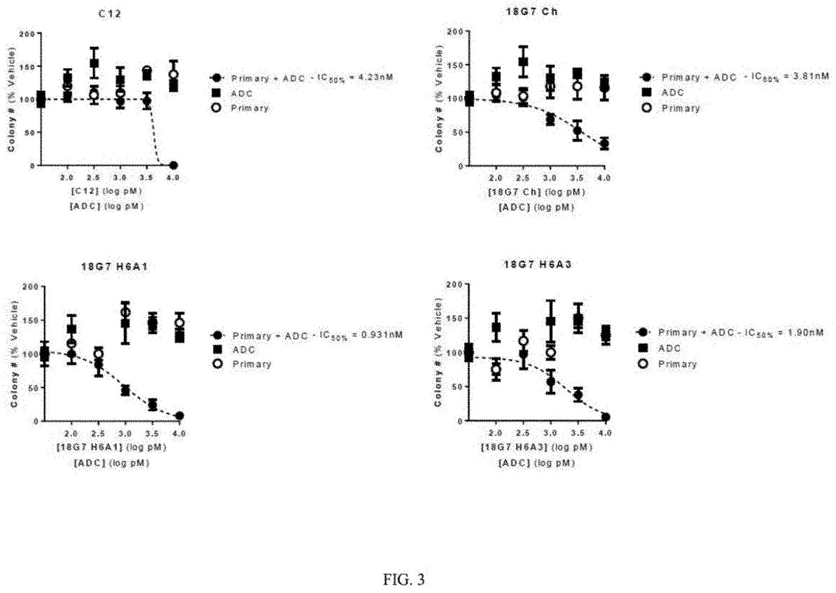

The results are depicted in FIG. 3 and TABLE 11 which show that each combination of primary antibody and ADC antibody had some activity to reduce the number of tumorspheres that formed over the period of the assay. The combination of the C12 antibody and the ADC antibody had substantially little activity to reduce tumorsphere formation at lower concentrations of the combination compared to the control. However, the combination of the C12 antibody and the ADC antibody was increasingly effective to inhibit tumorsphere formation at concentrations above 3 nM. The combination of the 18G7 H6A1 antibody and the ADC antibody had the lowest IC50 score of 0.931 nM.

TABLE 11

| Treatment | IC50 (nM) |
|---|---|
| C12 + ADC (CL-DMSA) | 4.23 |
| 18G7Ch + ADC (CL-DMSA) | 3.81 |
| 18G7H6A1 + ADC (CL-DMSA) | 0.931 |
| 18G7H6A3 + ADC (CL-DMSA) | 1.90 |

### Example 4-Tumorsphere Assay with Conjugated Anti-LGR5 Antibodies in Human Cells

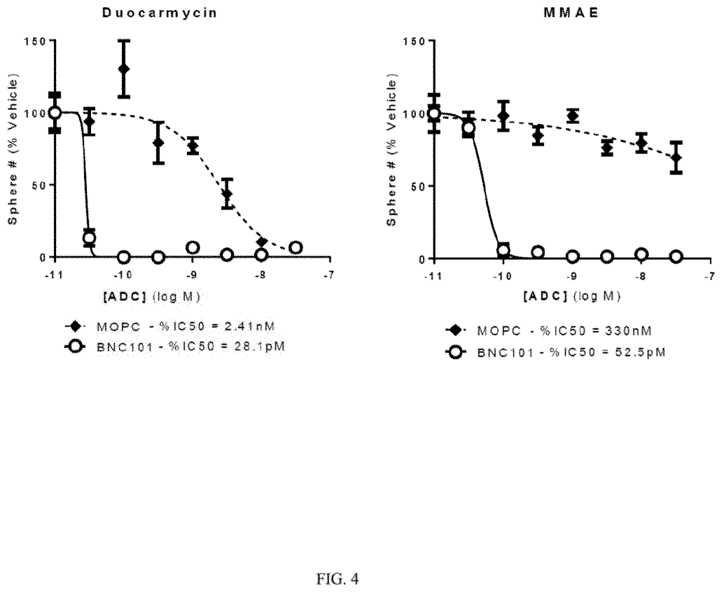

A tumorsphere assay was penned with human CT1 cells expressing LGR5 treated with either human anti-LGR5 antibody 18G7H6A3 (BNC101) conjugated to duocarmycin (DMSA) via a cleavable linker (CL-DMSA), or human anti-LGR5 antibody 18G7H6A3 (BNC101) conjugated to monomethyl auristatin E (MMAE) via a non-cleavable linker (NL-MMAE). CT1 cells are low passage primary cells derived from a patient with stage IV metastatic colon cancer that can be induced to form tumorspheres, and include mutations in the following genes: K-Ras, PI3K, PTEN, p53 and APC. Conjugated anti-LGR antibodies, and control MOPC antibody conjugated with CL-DMSA and NL-MMAE were prepared by Concortis Biotherapeutics, San Diego Calif. Cells were plated as described in Example 3, and treated with conjugated 18G7H6A3 or control MOPC antibody at 0.01, 0.03, 0.1, 0.3, 1, 3, 10, and 30 nM. After seven days, tumorspheres were counted. The results are depicted in FIG. 4 and TABLE 12 which show that the IC50 concentrations for 18G7H6A3 conjugated with either CL-DMSA or NL-MMAE were at least two orders of magnitude lower than the IC50 concentrations for control MOPC antibody conjugated with the same linker-drugs. The IC50 for 18G7H6A3 conjugated with CL-DMSA was lower than the IC50 for 18G7H6A3 conjugated with or NL-MMAE.

TABLE 12

| Treatment | IC50 |
|---|---|
| 18G7H6A3 conjugated with CL-DMSA | 28.1 pM |
| MOPC conjugated with CL-DMSA | 2.41 nM |
| 18G7H6A3 conjugated with NL-MMAE | 52.5 pM |
| MOPC conjugated with NL-MMAE | 330 nM |

### Example 5—In Vivo Treatment of a Xenograft Model with Anti-LGR5 Drug Conjugates

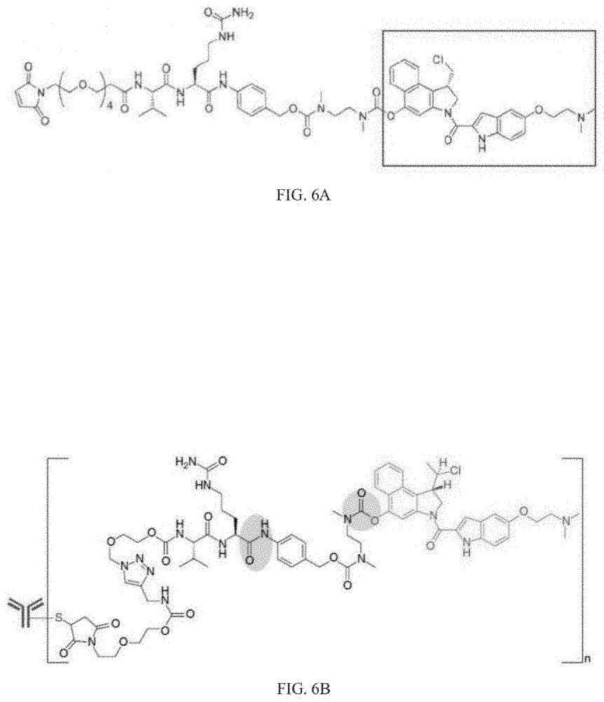

Seven-eight week old female CB.17 SCID mice (Charles River Laboratories) were inoculated with 2000 CT1 cells (1:1 matrigel:medium). Tumors developed for 25 days to an average volume of 120-130 mm³. Animals were administered different ADC treatments on days 25, 32 and 39. ADCs included 18G7H6A3 (BNC101) conjugated with DMSA or MMAE. Conjugated 18G7H6A3 with DMSA or MMAE were supplied by Concortis Biotherapeutics, San Diego Calif., and included vc-PAB conjugation using a Seattle Genetics conjugation technique. FIGS. 6A and 6B depict structures of a linker and a DMSA substantially similar to those used in this Example. In FIG. 6A, the structure include an MA-PEG4-vcPAB-diaminoethyl-carbamoyl-duocarmycin, with the DMSA shown in the box, in FIG. 6B, the DMSA is linked to an antibody. Control antibodies included MOPC antibody conjugated with DMSA or MMAE.

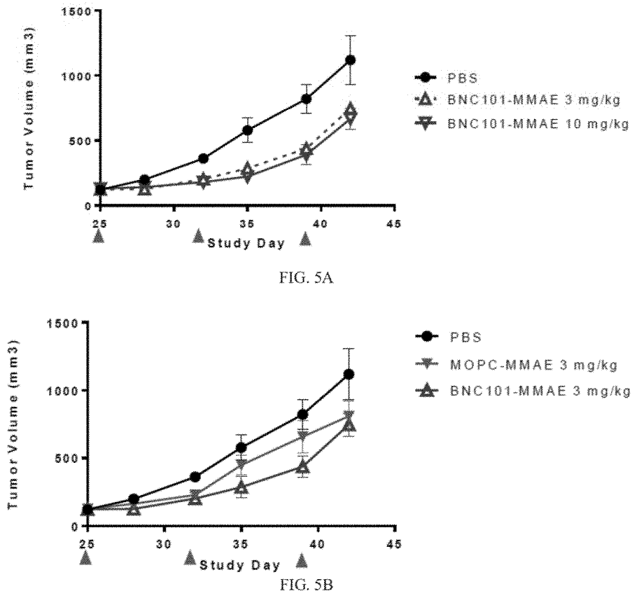

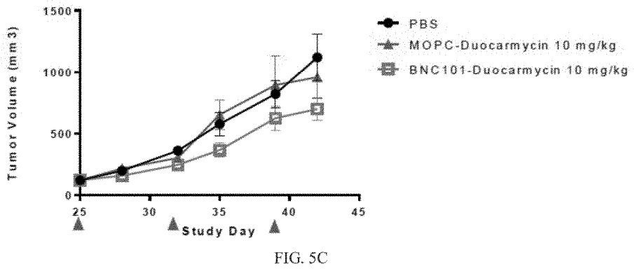

Treatment groups included: PBS; 3 mg/kg MOPC-Duocarmycin: 10 mg/kg MOPC-Duocarmycin; 3 mg/kg MOPC-MMAE; 10 mg/kg MOPC-MMAE; 3 mg/kg BNC101-Duocarmycin; 10 mg/kg BNC101-Duocarmycin; 3 mg/kg BNC101-MMAE; and 10 mg/kg BNC101-MMAE, (n=6). Tumours were measured every 3-4 days using automated digital calipers by taking an average of three measurements for tumour length and width. The experiment was terminated on day 42, and tumors were collected and dissociated for FACS and tumorsphere formation assays. Results are depicted in FIGS. 5A-5C. FIG. 5A shows that treatment with 18G7H6A3 conjugated with MMAE inhibited tumor growth compared to treatment with PBS control by about at least 37% at day 42. FIGS. 5B and 5C show that treatment with either 18G7H6A3 conjugated with MMAE (FIG. 5B), or 18G7H6A3 conjugated with duocarmycin (FIG. 5C) was effective to inhibit tumor growth for at least 40 days compared to treatments with conjugated MOPC controls.

### Certain Embodiments

Certain embodiments provided herein include the following aspects.

Aspect 1. An antibody drug conjugate comprising an antibody or antigen-binding fragment thereof that specifically binds to human leucine-rich repeat containing G-protein-coupled receptor 5 (LGR5), wherein the antibody or antigen-binding fragment thereof is conjugated to a drug via a linker.

Aspect 2. The antibody drug conjugate of aspect 1, wherein the antibody or antigen-binding fragment thereof comprises: a heavy chain complementary determining region (CDR1) comprising SEQ ID NO:23, or conservative variations thereof, a heavy chain complementary determining region 2 (CDR2) comprising SEQ ID NO:25, or conservative variations thereof, a heavy chain complementary determining region 3 (CDR3) comprising SEQ ID NO:27, or conservative variations thereof, a light chain CDR1 comprising SEQ ID NO:29, or conservative variations thereof, a light chain CDR2 comprising SEQ ID NO:31, or conservative variations thereof, and a light chain CDR3 comprising SEQ ID NO:33, or conservative variations thereof.

Aspect 3. The antibody drug conjugate of aspect 1, wherein the antibody or antigen-binding fragment thereof comprises a heavy chain CDR1 comprising SEQ ID NO:23.

Aspect 4. The antibody drug conjugate of any one of aspects 1-3, wherein the anti-LGR5 antibody or antigen-binding fragment thereof comprises an IgG1.

Aspect 5. The antibody drug conjugate of any one of aspects 1-4, wherein the linker is a non-cleavable linker.

Aspect 6. The antibody drug conjugate of any one of aspects 1-4, wherein the linker is a cleavable linker.

Aspect 7. The antibody drug conjugate of any one of aspects 1-6, wherein the drug is selected from a microtubulin inhibitor and a DNA damaging agent.

Aspect 8. The antibody drug conjugate of aspect 7, wherein the microtubule inhibitor is selected form the group consisting of cabazitaxel, colcemid, colchicine, cryptophycin, demecolcine, docetaxel, 2-Methoxyestradiol, docodazole, paclitaxel, taccalonolide, taxane, and vinblastine.

Aspect 9. The antibody drug conjugate of any one of aspects 1-6, wherein the drug is selected from the group consisting of monomethyl auristatin F, monomethyl auristatin E, monomethyl dolastatin 10, duocarmycin, maytansanoid 1, dualstatin 3, calicheamicin, and duocamycin.

Aspect 10. A pharmaceutical composition comprising the antibody drug conjugate of any one of aspects 1-9 and a pharmaceutically acceptable carrier.

Aspect 11. A method of treating a subject having a cancer comprising administering an effective amount of the antibody drug conjugate of any one of aspects 1-9 to the subject in need thereof.

Aspect 12. The method of aspect 11, wherein the cancer comprises a solid tumor.

Aspect 13. The method of aspect 11, wherein the cancer comprises a cancer stem cell.

Aspect 14. The method of aspect 11, wherein the cancer is selected from the group consisting of: lung cancer, breast cancer, colon cancer, and pancreatic cancer.

Aspect 15. The method of aspect 11, wherein the cancer comprises a cell selected from the group consisting of: a triple negative breast cancer cell, a colon cancer cell having a mutation in a gene selected from the group consisting of K-Ras, H-Ras, APC, PI3K, PTEN, STK11, RB1, TP53, FGFR2, VANGL2, and ISCO, and a small cell lung cancer cell.

Aspect 16. The method of any one of aspects 11-15, wherein the subject is mammalian.

Aspect 17. The method of any one of aspects 11-16, wherein the subject is human.

Aspect 18. The method of any one of aspects 11-17, comprising administering an additional therapy in combination with the antibody drug conjugate, wherein the additional therapy is selected from the group consisting of: radiotherapy, and a chemotherapeutic agent.

Aspect 19. The method of aspect 18, wherein administration of the antibody drug conjugate is concurrent with administration of the additional therapy

Aspect 20. The method of aspect 18, wherein the chemotherapeutic agent is selected from the group consisting of: folinic acid, fluorouracil, irinotecan, gemcitabine, paclitaxel, nab-paclitaxel, ERBITUX (cetuximab), PI3K/mTOR dual inhibitor (NVP), and SN38.

Aspect 21. The method of aspect 18, wherein the additional therapy comprises folinic acid, fluorouracil, and irinotecan.

Aspect 22. A method of preparing the antibody drug conjugate of any one of aspects 1-9 comprising: linking the linker to the drug; and conjugating the linked drug to the antibody.

Aspect 23. The method of aspect 22, comprising purifying the conjugated antibody.

The term “comprising” as used herein is synonymous with “including.” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. An antibody drug conjugate comprising an antibody or antigen-binding fragment thereof that specifically binds to human leucine-rich repeat containing G-protein-coupled receptor 5 (LGR5), wherein the antibody or antigen-binding fragment thereof is conjugated to a drug via a linker, wherein the antibody or antigen-binding fragment thereof comprises:
a heavy chain complementary determining region (CDR1) comprising SEQ ID NO:23, or conservative variations thereof,
a heavy chain complementary determining region 2 (CDR2) comprising SEQ ID NO:25, or conservative variations thereof,
a heavy chain complementary determining region 3 (CDR3) comprising SEQ ID NO:27, or conservative variations thereof,
a light chain CDR1 comprising SEQ ID NO:29, or conservative variations thereof,
a light chain CDR2 comprising SEQ ID NO:31, or conservative variations thereof, and
a light chain CDR3 comprising SEQ ID NO:33, or conservative variations thereof.

2. The antibody drug conjugate of claim 1, wherein the antibody or antigen-binding fragment thereof comprises a heavy chain CDR1 comprising SEQ ID NO:23.

3. The antibody drug conjugate of claim 1, wherein the anti-LGR5 antibody or antigen-binding fragment thereof comprises an IgG1.

4. The antibody drug conjugate of claim 1, wherein the linker is a non-cleavable linker.

5. The antibody drug conjugate of claim 1, wherein the linker is a cleavable linker.

6. The antibody drug conjugate of claim 1, wherein the drug is selected from a microtubulin inhibitor and a DNA damaging agent.

7. The antibody drug conjugate of claim 6, wherein the microtubule inhibitor is selected form the group consisting of cabazitaxel, colcemid, colchicine, cryptophycin, demecolcine, docetaxel, 2-Methoxyestradiol, docodazole, paclitaxel, taccalonolide, taxane, and vinblastine.

8. The antibody drug conjugate of claim 1, wherein the drug is selected from the group consisting of monomethyl auristatin F, monomethyl auristatin E, monomethyl dolastatin 10, duocarmycin, maytansanoid 1, dualstatin 3, calicheamicin, and duocamycin.

9. A pharmaceutical composition comprising the antibody drug conjugate of claim 1 and a pharmaceutically acceptable carrier.

10. A method of treating a subject having a cancer comprising administering an effective amount of the antibody drug conjugate of claim 1 to the subject in need thereof.

11. The method of claim 10, wherein the cancer comprises a solid tumor.

12. The method of claim 10, wherein the cancer comprises a cancer stem cell.

13. The method of claim 10, wherein the cancer is selected from the group consisting of: lung cancer, breast cancer, colon cancer, and pancreatic cancer.

14. The method of claim 10, wherein the cancer comprises a cell selected from the group consisting of: a triple negative breast cancer cell, a colon cancer cell having a mutation in a gene selected from the group consisting of K-Ras, H-Ras, APC, PI3K, PTEN, STK11, RB1, TP53, FGFR2, VANGL2, and ISCO, and a small cell lung cancer cell.

15. The method of claim 10, wherein the subject is mammalian.

16. (canceled)

17. The method of claim 10, comprising administering an additional therapy in combination with the antibody drug conjugate, wherein the additional therapy is selected from the group consisting of: radiotherapy, and a chemotherapeutic agent.

18. The method of claim 17, wherein administration of the antibody drug conjugate is concurrent with administration of the additional therapy

19. The method of claim 17, wherein the chemotherapeutic agent is selected from the group consisting of: folinic acid, fluorouracil, irinotecan, gemcitabine, paclitaxel, nab-paclitaxel, ERBITUX (cetuximab), PI3K mTOR dual inhibitor (NVP), and SN38.

20. The method of claim 17, wherein the additional therapy comprises folinic acid, fluorouracil, and irinotecan.

21. A method of preparing the antibody drug conjugate of claim 1 comprising:
linking the linker to the drug; and
conjugating the linked drug to the antibody.

22. (canceled)
